(19) United States
(12) Patent Application Publication
(10) Pub. No.: US 2009/0137822 A1
(43) Pub. Date: May 28, 2009

(54) Process for preparing 3-substituted thiophene

(76) Inventors: Akio Matsushita, Yamaguchi (JP); Kiyotaka Yoshii, Yamaguchi (JP); Mizuho Oda, Yamaguchi (JP); Masayoshi Oue, Yamaguchi (JP); Shuji Yamada, Yamaguchi (JP)

Correspondence Address: BIRCH STEWART KOLASCH & BIRCH, PO BOX 747, FALLS CHURCH, VA, 22040-0747, US

(21) Appl. No.: 11/920,568

(22) PCT Filed: May 16, 2006
(86) PCT No.: PCT/JP2006/309726

§ 371 Date: Nov. 16, 2007

(30) Foreign Application Priority Data
May 16, 2005 (JP) 2005-142212
May 16, 2005 (JP) 2005-142213
Jun. 8, 2005 (JP) 2005-168190
Sep. 22, 2005 (JP) 2005-276195

(51) Int. Cl. C07D 333/38; C07D 333/22; C07D 333/16

(52) U.S. Cl. 549/61; 549/71; 549/72; 549/78

(57) ABSTRACT

The present invention relates to a process for preparing a 3-substituted thiophene represented by the formula (2):

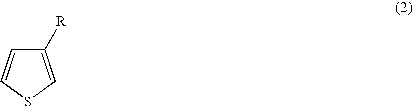

wherein R represents a cyano group, a formyl group, a carboxyl group, a hydrocarbyloxycarbonyl group which may have a substituent(s) or an acyl group which may have a substituent(s),
   which comprises reacting a vinyl compound represented by the formula (1):

RCH═CHY (1)
wherein R has the same meaning as defined above, and
Y represents a leaving group,
   and an α-mercaptoacetaldehyde or a multimer thereof.

### TECHNICAL FIELD

The present invention relates to a process for preparing a 3-substituted thiophene. The 3-substituted thiophene is a compound useful, for example, as a synthetic intermediate or a starting material for a medicine, an agricultural chemical, etc., a synthetic starting material of chemicals for photography, etc.

### BACKGROUND ART

Heretofore, as a process for preparing a 3-substituted thiophene, for example, the following methods are disclosed.

(1) There is disclosed a method in which methyl acrylate and 2,5-dihydroxy-1,4-dithiane are reacted in the presence of sodium carbonate in acetonitrile to prepare a crude product of 3-hydroxy-4-methoxycarbonyltetrahydrothiophene, then, the crude product of 3-hydroxy-4-methoxycarbonyltetrahydrothiophene and methanesulfonyl chloride are reacted in the presence of triethylamine in toluene to obtain a toluene solution of 3-methoxycarbonyl-2,5-dihydrothiophene, further, sulfuryl chloride is added to the toluene solution to carry out the reaction to prepare 3-methoxycarbonylthiophene in a total isolation yield of 54.4% (for example, see patent literature 1).
(2) There is disclosed a method in which 2,5-dihydroxy-1,4-dithiane and acrolein are reacted in water to obtain 2,5-dihydrothiophene-3-carboxaldehyde, then, the 2,5-dihydrothiophene-3-carboxaldehyde and sulfuryl chloride are reacted in 1,2-dichloroethane to prepare 3-formylthiophene in an overall reaction yield of 62.8% (for example, see patent literature 2).
(3) There is disclosed a method in which 3-formylthiophene synthesized by the existing method and hydroxylamine hydrochloride are reacted in N-methyl-2-pyrrolidone to prepare 3-cyanothiophene (for example, see patent literature 3).

However, in either of the above-mentioned methods, multi steps in different reaction systems are required, and accompanied thereby, operations or post-treatments are complicated so that they are not at all effective methods in the aspect of an industrial preparation process.

(4) There is disclosed a method in which β-thiophenecarbaldehyde and diazomethane are reacted for 12 hours to prepare 3-acetylthiophene (for example, see Non-patent literature 1). However, according to this method, there are problems that a reaction time is long and diazomethane which is industrially difficult in handling must be used.
(5) There is disclosed a method in which 3-ethylthiophene and bromine are reacted to prepare 3-bromoethylthiophene, then, the product is hydrolyzed and oxidized with lead acetate to prepare 3-acetylthiophene (for example, see Non-patent literature 2). However, according to this method, there is a problem that a bromine gas which is industrially difficult in handling or a lead compound having high toxicity must be used.
(6) There is disclosed a method in which 3-bromothiophene and n-butyl lithium are reacted, then, acetoaldehyde is reacted to the product to prepare 1-(3-thienyl)ethanol, and the product is oxidized with lead acetate to prepare 3-acetylthiophene (for example, see Non-patent literature 3). However, according to this method, there are problems that n-butyl lithium which is industrially difficult in handling or a lead compound having high toxicity must be used.
(7) There is disclosed a method in which thiophene-3-carbonyl chloride and an organic cadmium are reacted to prepare acylthiophene or benzoylthiophene (for example, see Non-patent literature 4). However, according to this method, an organic cadmium compound having high toxicity must be used.

That is, in either of the above-mentioned methods, there are various problems and they are not an industrially satisfiable method.

(8) There is disclosed a method in which 3-acetylthiophene and diphenyl diselenide are reacted in methanol to synthesize 3-(2,2-dimethoxyacetyl)-thiophene (for example, see Non-patent literature 5). Also, there is disclosed a method in which 3-acetylthiophene is reacted with a hydrogen chloride gas and methyl nitrite to synthesize 3-(2,2-dimethoxyacetyl)-thiophene (for example, see patent literature 4). However, according to these methods, there is a problem that expensive 3-acetylthiophene must be used as a starting material.
Patent literature 1: JP 2003-206286 A
Patent literature 2: JP 2001-199979 A
Patent literature 3: JP 2001-233873 A
Patent literature 4: U.S. Pat. No. 5,159,117
Non-patent literature 1: Chem. Ber. 98, 3187 (1965)
Non-patent literature 2: Tetrahedron Lett., 52, 4705 (1975)
Non-patent literature 3: J. Org. Chem., 43(8), 1591 (1978)
Non-patent literature 4: J. Am. Chem. Soc., 77, 5365 (1955)
Non-patent literature 5: J. Org. Chem., 55(15), 4523 (1990)

### DISCLOSURE OF THE INVENTION

### Problems to be Solved by the Invention

An object of the present invention is to solve the above-mentioned problems and to provide an industrially suitable process for preparing a 3-substituted thiophene which is capable of producing 3-substituted thiophene with a simple and convenient method under mild conditions, with a single step and high yield.

### Means to Solve the Problems

The present invention is to provide a process for preparing a 3-substituted thiophene represented by the formula (2):

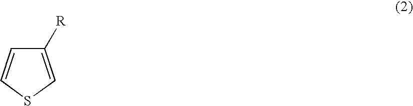

wherein R represents a cyano group, a formyl group, a carboxyl group, a hydrocarbyloxycarbonyl group which may have a substituent(s) or an acyl group which may have a substituent(s),
   which comprises reacting a vinyl compound represented by the formula (1):

RCH═CHY (1)
wherein R has the same meaning as defined above, and
Y represents a leaving group,
   and an α-mercaptoacetaldehyde or a multimer thereof.

### EFFECTS OF THE INVENTION

According to the present invention, an industrially suitable process for preparing a 3-substituted thiophene which is capable of producing a 3-substituted thiophene with a simple and convenient method under mild conditions with high yield can be provided.

### BEST MODE FOR CARRYING OUT THE INVENTION

The vinyl compound to be used in the reaction of the present invention is represented by the above-mentioned formula (1). In the formula (1), R represents a cyano group, a formyl group, a carboxyl group, a hydrocarbyloxycarbonyl group which may have a substituent(s), or an acyl group which may have a substituent(s). As the hydrocarbyloxycarbonyl group, there may be mentioned, for example, a straight or branched alkoxycarbonyl group having 2 to 6 carbon atoms such as a methoxycarbonyl group, ethoxycarbonyl group, propoxycarbonyl group, butoxycarbonyl group, pentyloxycarbonyl group, etc.; an aralkyloxycarbonyl group having 8 to 21 carbon atoms such as a benzyloxycarbonyl group, etc.; an aryloxycarbonyl group having 7 to 21 carbon atoms such as a phenoxycarbonyl group, etc., and the like. Also, as the acyl group, there may be mentioned, for example, a straight or branched alkylcarbonyl group having 2 to 9 carbon atoms such as an acetyl group, propionyl group, butyryl group, valeryl group, hexanoyl, heptanoyl group, octanoyl group, etc.; an aralkylcarbonyl group having 8 to 21 carbon atoms such as a benzylcarbonyl group, etc.; an arylcarbonyl group having 7 to 21 carbon atoms such as a benzoyl group, etc., and the like. Of these, preferred are a cyano group, formyl group, a straight or branched alkoxycarbonyl group having 2 to 4 carbon atoms and a straight or branched acyl group having 2 to 9 carbon atoms, more preferably a cyano group, formyl group, methoxycarbonyl group, acetyl group, benzoyl group, valeryl group, octanoyl group and 2,2-dimethoxyacetyl group.

The above-mentioned hydrocarbyloxycarbonyl group and acyl group may have a substituent(s), and as the substituent(s), there may be mentioned, for example, a substituent(s) formed through a carbon atom, a substituent(s) formed through an oxygen atom, a substituent(s) formed through a nitrogen atom, a substituent(s) formed through a sulfur atom, a halogen atom, etc. Incidentally, a number of the substituent(s) is not particularly limited.

As the above-mentioned substituent(s) formed through a carbon atom, there may be mentioned, for example, a straight or branched alkyl group having 1 to 20 carbon atoms such as a methyl group, ethyl group, propyl group, butyl group, pentyl group, hexyl group, heptyl group, octyl group, nonyl group, decyl group, undecyl group, dodecyl group, etc.; a cycloalkyl group having 3 to 7 carbon atoms such as a cyclopropyl group, cyclobutyl group, cyclopentyl group, cyclohexyl group, cycloheptyl group, etc.; a straight or branched alkenyl group having 2 to 7 carbon atoms such as a vinyl group, allyl group, propenyl group, cyclopropenyl group, cyclobutenyl group, cyclopentenyl group, etc.; a heterocyclic group having at least one hetero atom selected from an oxygen atom, nitrogen atom and sulfur atom such as a quinolyl group, pyridyl group, pyrrolidyl group, pyrrolyl group, furyl group, thienyl group, etc.; an aryl group having 6 to 20 carbon atoms such as a phenyl group, tolyl group, fluorophenyl group, xylyl group, biphenyl group, naphthyl group, anthryl group, phenanthryl group, etc.; an aralkyl group having 7 to 10 carbon atoms such as a benzyl group, phenethyl group, phenylpropyl group, etc.; an acyl group (which may be in an acetal form) having 2 to 11 carbon atoms such as an acetyl group, propionyl group, acryloyl group, pivaloyl group, cyclohexylcarbonyl group, benzoyl group, naphthoyl group, toluoyl group, etc.; carboxyl group; a straight or branched alkoxycarbonyl group having 2 to 7 carbon atoms such as a methoxycarbonyl group, ethoxycarbonyl group, etc.; an aryloxycarbonyl group having 7 to 20 carbon atoms such as a phenoxycarbonyl group, etc.; and a halogenated alkyl group in which a straight or branched alkyl group having 1 to 6 carbon atoms is substituted by at least one halogen atom such as a trifluoromethyl group, etc. Incidentally, these groups may contain various kinds of isomers.

As the above-mentioned substituent(s) formed through an oxygen atom, there may be mentioned, for example, hydroxyl group; a straight or branched alkoxyl group having 1 to 7 carbon atoms such as a methoxyl group, ethoxyl group, propoxyl group, butoxyl group, pentyloxyl group, hexyloxyl group, heptyloxyl group, etc.; an aralkyloxyl group having 7 to 20 carbon atoms such as a benzyloxyl group, etc.; and an aryloxyl group having 6 to 20 carbon atoms such as a phenoxyl group, toluoyloxyl group, naphthyloxyl group, etc. Incidentally, these groups may contain various kinds of isomers.

As the above-mentioned substituent(s) formed through a nitrogen atom, there may be mentioned, for example, a primary amino group which has a straight or branched alkyl group (which may form a ring) having 1 to 6 carbon atoms or an aryl group having 6 to 20 carbon atoms such as a methylamino group, ethylamino group, butylamino group, cyclohexylamino group, phenylamino group, naphthylamino group, etc.; a secondary amino group having two of the above-mentioned substituents such as a dimethylamino group, diethylamino group, dibutylamino group, methylethylamino group, methylbutylamino group, diphenylamino group, N-methyl-N-methanesulfonylamino group, etc.; a heterocyclic series amino group such as a morpholino group, piperidino group, piperazinyl group, pyrazolidinyl group, pyrrolidino group, indolyl group, etc.; and an imino group. Incidentally, these groups may contain various kinds of isomers.

As the above-mentioned substituent(s) formed through a sulfur atom, there may be mentioned, for example, a mercapto group; an alkylthio group such as a methylthio group, ethylthio group, propylthio group, etc.; and an arylthio group such as phenylthio group, toluoylthio group, naphthylthio group, etc. Incidentally, these groups may contain various kinds of isomers.

As the above-mentioned halogen atom, there may be mentioned a fluorine atom, chlorine atom, bromine atom and iodine atom.

In the formula (1), Y represents a leaving group (an eliminatable group), and there may be mentioned, for example, a substituted amino group such as a monoalkylamino group, monoarylamino group, dialkylamino group, diarylamino group, etc.; [NR¹R²R³]⁺X⁻ group (wherein R¹ to R³ may be the same or different from each other, and represent an alkyl group having 1 to 4 carbon atoms, phenyl group or benzyl group, and X represents a halogen atom); a halogen atom such as a fluorine atom, chlorine atom, bromine atom, iodine atom, etc.; a substituted thio group such as an alkylthio group, arylthio group, etc.; an alkylsulfonyl group such as a mesyl group, etc.; an arylsulfonyl group such as a benzenesulfonyl group, tosyl group, etc.; an alkylsulfonyloxyl group such as a methanesulfonyloxyl group, ethanesulfonyloxyl group, etc.; an arylsulfonyloxyl group such as a benzenesulfonyloxyl group, p-toluenesulfonyloxyl group, etc.; an acyloxyl group having 2 to 7 carbon atoms such as an acetoxyl group, propionyloxyl group, benzoyloxyl group, etc.; and a hydrocarbyloxyl group which may have a substituent(s), preferably a dialkylamino group, diarylamino group, a halogen atom and a hydrocarbyloxyl group which may have a substituent(s), particularly preferably a dialkylamino group, or a hydrocarbyloxyl group which may have a substituent(s).

As the above-mentioned hydrocarbyloxyl group which may have a substituent(s), there may be mentioned, for example, a straight or branched alkoxyl group having 1 to 5 carbon atoms such as a methoxyl group, ethoxyl group, propoxyl group, butoxyl group, pentyloxyl group, etc.; a cycloalkoxyl group having 3 to 7 carbon atoms such as a cyclopropoxyl group, cyclobutoxyl group, cyclopentyloxyl group, cyclohexyloxyl group, cycloheptyloxyl group, etc.; an aralkyloxyl group having 7 to 20 carbon atoms such as a benzyloxyl group, phenethyloxyl group, phenylpropoxyl group, etc.; an aryloxyl group having 6 to 20 carbon atoms such as a phenoxyl group, naphthoxyl group, anthoxyl group, etc., preferably an alkoxyl group having 1 to 6 carbon atoms. Incidentally, these groups may contain various kinds of isomers.

Incidentally, the vinyl compound represented by the above-mentioned formula (1) wherein R is a 2,2-dihydrocarbyloxyacetyl group, and Y is an alkoxyl group can be obtained as shown by the following reaction scheme (1):

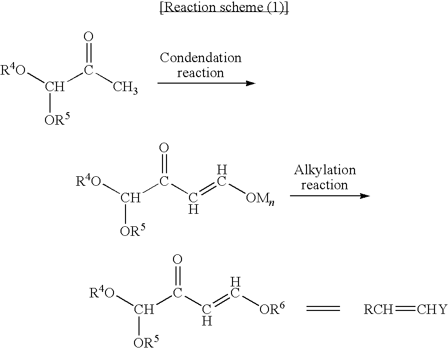

wherein R⁴ and R⁵ each represent a hydrocarbon group which may have a substituent(s), R⁶ represents an alkyl group, M represents an alkali metal atom or an alkaline earth metal atom, and n is 1 or ½,
   by subjecting 1,1-dihydrocarbyloxy-2-propanone and a formic acid ester to condensation reaction, and then, reacting an alkylating agent with the resulting compound (described in Reference examples 6 to 7 mentioned below).

The above-mentioned R⁴ and R⁵ are hydrocarbon groups which may have a substituent(s), and more specifically, there may be mentioned, for example, an alkyl group such as a methyl group, ethyl group, propyl group, butyl group, pentyl group, hexyl group, heptyl group, octyl group, nonyl group, decyl group, undecyl group, dodecyl group, etc.; a cycloalkyl group such as a cyclopropyl group, cyclobutyl group, cyclopentyl group, cyclohexyl group, cycloheptyl group, etc.; an aralkyl group such as a benzyl group, phenethyl group, phenylpropyl group, etc.; an aryl group such as a phenyl group, naphthyl group, anthryl group, etc. Incidentally, these groups may contain various kinds of isomers. Incidentally, R⁴ and R⁵ may be bonded to each other to form a ring.

The above-mentioned hydrocarbon group may have a substituent (s). As the substituent (s), there may be mentioned a substituent(s) formed through a carbon atom, a substituent(s) formed through an oxygen atom, a substituent(s) formed through a nitrogen atom, a substituent(s) formed through a sulfur atom, a halogen atom, etc.

As the above-mentioned substituent(s) formed through a carbon atom, there may be mentioned, for example, an alkyl group such as a methyl group, ethyl group, propyl group, butyl group, pentyl group, hexyl group, etc.; a cycloalkyl group such as a cyclopropyl group, cyclobutyl group, cyclopentyl group, cyclohexyl group, cycloheptyl group, etc.; an alkenyl group such as a vinyl group, allyl group, propenyl group, cyclopropenyl group, cyclobutenyl group, cyclopentenyl group, etc.; a heterocyclic group such as a quinolyl group, pyridyl group, pyrrolidyl group, pyrrolyl group, furyl group, thienyl group, etc.; an aryl group such as a phenyl group, tolyl group, fluorophenyl group, xylyl group, biphenyl group, naphthyl group, anthryl group, phenanthryl group, etc.; an acyl group (which may be in an acetal form) such as an acetyl group, propionyl group, acryloyl group, pivaloyl group, cyclohexylcarbonyl group, benzoyl group, naphthoyl group, toluoyl group, etc.; a carboxyl group; an alkoxycarbonyl group such as a methoxycarbonyl group, ethoxycarbonyl group, etc.; an aryloxycarbonyl group such as a phenoxycarbonyl group, etc.; a halogenated alkyl group such as a trifluoromethyl group, etc.; and a cyano group. Incidentally, these groups may contain various kinds of isomers.

As the above-mentioned substituent(s) formed through an oxygen atom, there may be mentioned, for example, a hydroxyl group; an alkoxyl group such as a methoxyl group, ethoxyl group, propoxyl group, butoxyl group, pentyloxyl group, hexyloxyl group, heptyloxyl group, benzyloxyl group, etc.; and an aryloxyl group such as a phenoxyl group, toluoyloxyl group, naphthyloxyl group, etc. Incidentally, these groups may contain various kinds of isomers.

As the above-mentioned substituent(s) formed through a nitrogen atom, there may be mentioned, for example, a primary amino group such as a methylamino group, ethylamino group, propylamino group, butylamino group, cyclohexylamino group, phenylamino group, naphthylamino group, etc.; a secondary amino group such as a dimethylamino group, diethylamino group, dipropylamino group, dibutylamino group, methylethylamino group, methylpropylamino group, methylbutylamino group, diphenyl amino group, N-methyl-N-methanesulfonylamino group, etc.; a heterocyclic amino group such as a morpholino group, piperidino group, piperazinyl group, pyrazolidinyl group, pyrrolidino group, indolyl group etc.; and an imino group. Incidentally, these groups may contain various kinds of isomers.

As the above-mentioned substituent(s) formed through a sulfur atom, there may be mentioned, for example, a mercapto group; a thioalkoxyl group such as a thiomethoxyl group, thioethoxyl group, thiopropoxyl group, etc.; a thioaryloxyl group such as a thiophenoxyl group, thiotoluoyloxyl group, thionaphthyloxyl group, etc. Incidentally, these groups may contain various kinds of isomers.

As the above-mentioned halogen atom, there may be mentioned a fluorine atom, chlorine atom, bromine atom and iodine atom.

Also, R⁶ is an alkyl group, and this is the same as those defined in the above-mentioned R⁴ and R⁵.

As the α-mercaptoacetaldehyde or a multimer thereof to be used in the reaction of the present invention, there may be suitably used, for example, 1,4-dithiane-2,5-diol which is a stable dimer.

An amount of the above-mentioned α-mercaptoacetaldehyde or a multimer thereof to be used is preferably 0.2 to 20 mols, more preferably 0.5 to 10 mols in terms of the α-mercaptoacetaldehyde based on 1 mol of the vinyl compound.

The reaction of the present invention is desirably carried out in the presence of a solvent. As the solvent to be used, it is not specifically limited so long as it does not inhibit the reaction, and there may be mentioned, for example, water: an alcohol such as methanol, ethanol, n-propyl alcohol, isopropyl alcohol, n-butyl alcohol, isobutyl alcohol, t-butyl alcohol, etc.; an amide such as N,N-dimethylformamide, N,N-dimethylacetamide, N-methyl-2-pyrrolidone, etc.; a urea such as N,N′-dimethyl-2-imidazolidinone, etc.; a nitrile such as acetonitrile, propionitrile, benzonitrile, etc.; an aromatic hydrocarbon such as benzene, toluene, xylene, cumene, etc.; a halogenated aliphatic hydrocarbon such as methylene chloride, 1,2-dichloroethane, 1,1-dichloroethane, etc.; a halogenated aromatic hydrocarbon such as chlorobenzene, etc.; an ether such as diethyl ether, diisopropyl ether, tetrahydrofuran, etc., preferably an alcohol, an amide, a nitrile, an aromatic hydrocarbon, a halogenated aliphatic hydrocarbon, a halogenated aromatic hydrocarbon, an ether, more preferably methanol, ethanol, t-butyl alcohol, N,N-dimethylformamide, acetonitrile, propionitrile, toluene, 1,2-dichloroethane, chlorobenzene and tetrahydrofuran are used. Incidentally, these solvents may be used alone or in admixture of two or more kinds.

An amount of the above-mentioned solvent to be used may be optionally adjusted depending on a degree of uniformity or condition of stirring of the reaction mixture, and preferably 1 to 100 ml, more preferably 2 to 50 ml based on 1 g of the vinyl compound.

The reaction of the present invention is carried out, for example, by mixing the vinyl compound, α-mercaptoacetaldehyde or a multimer thereof, and a solvent, and stirring the mixture, etc. A reaction temperature at this time is preferably −10 to 200° C., more preferably 0 to 150° C., and a reaction pressure is not particularly limited.

Incidentally, in the reaction of the present invention, it is desirably carried out in the presence of an additive to raise activity of the reaction, and as the additive to be used, there may be mentioned, for example, an organic base such as triethylamine, pyridine, 1,8-diazabicyclo[5.4.0]-7-undecene, 1,5-diazabicyclo[4.3.0]-5-nonene, 1,4-diazabicyclo[2.2.2]octane, etc.; an inorganic base such as lithium carbonate, sodium carbonate, potassium carbonate, rubidium carbonate, cesium carbonate, sodium hydrogen carbonate, potassium hydrogen carbonate, sodium hydroxide, potassium hydroxide, etc.; a metal alcoholate such as sodium methoxide, sodium ethoxide, potassium t-butoxide, potassium n-butoxide, etc.; an organic acid such as acetic acid, propionic acid, methanesulfonic acid, p-toluenesulfonic acid, etc.; a Lewis acid such as titanium tetrachloride, tin tetrachloride, boron trifluoride (which may form a complex with an ether, methanol, n-propyl alcohol, water, acetic acid, ethylamine, tetrahydrofuran, etc.), titanium tetraisopropoxide, magnesium chloride, aluminum chloride, zinc chloride, etc., a mineral acid such as hydrochloric acid, sulfuric acid, etc., a cyclic polyether such as 15-crown 5 ether, 18-crown 6-ether, etc., a non-cyclic polyether such as polyethylene glycol dialkyl ether, etc., a quaternary ammonium salt such as tetramethyl ammonium chloride, tetraethyl ammonium chloride, tetrabutyl ammonium bromide, tetrabutyl ammonium iodide, benzyltrimethyl ammonium chloride, ALIQUAT 336, etc., a halogenated alkali such as lithium chloride, lithium bromide, lithium iodide, sodium bromide, sodium iodide, potassium bromide, potassium iodide, etc., preferably used are 1,8-diazabicyclo[5.4.0]-7-undecene, potassium carbonate, cesium carbonate, rubidium carbonate, titanium tetrachloride, tin tetrachloride, boron trifluoride diethyl ether complex, magnesium chloride, zinc chloride, hydrochloric acid, sulfuric acid, p-toluenesulfonic acid.

Incidentally, these additives may be used alone or in admixture of two or more kinds (when two or more kinds of additives are used, they may be added simultaneously or added by dividing them into several portions).

An amount of the above-mentioned additives to be used is preferably 0.01 to 10 mols, more preferably 0.02 to 5 mols based on 1 mol of the vinyl compound represented by the formula (1).

Specific examples of the 3-substituted thiophene represented by the formula (2) obtained by the present invention may be mentioned, for example, 3-cyanothiophene, 3-formylthiophene, 3-methoxycarbonylthiophene, 3-acetylthiophene, 3-benzoylthiophene, 3-valerylthiophene, 3-octanoylthiophene, 3-(2,2-dimethoxyacetyl)-thiophene, etc.

The 3-substituted thiophene obtained by the reaction of the present invention can be isolated and purified, after completion of the reaction, according to the conventional manner such as neutralization, extraction, filtration, concentration, distillation, recrystallization, column chromatography, etc.

### EXAMPLES

Next, the present invention will be explained more specifically by referring to Examples, but the scope of the present invention is not limited by these.

### Example 1

### [R=Methoxycarbonyl Group]; Synthesis of 3-methoxycarbonylthiophene

In a flask having an inner volume of 25 ml and equipped with a stirrer, a thermometer and a reflux condenser were charged 10 ml of 1,2-dichloroethane, 1.16 g (10.0 mmol) of methyl 3-methoxyacrylate and 0.91 g of 1,4-dithiane-2,5-diol (12 mmol as α-mercaptoacetaldehyde), then, 0.38 g (2 mmol) of titanium tetrachloride was gradually added dropwise thereto while maintaining the liquid temperature to 20° C., and the mixture was reacted under stirring at 67° C. for 2 hours. After completion of the reaction, the reaction mixture was filtered, and the filtrate was concentrated under reduced pressure. The concentrate was purified by silica gel column chromatography (Eluent; n-hexane/ethyl acetate=5/1 to 1/1 (volume ratio)) to obtain 0.62 g (Isolation yield; 44%) of 3-methoxycarbonylthiophene as colorless powder.

Physical properties of the 3-methoxycarbonylthiophene are as follows.

¹H-NMR (CDCl₃, δ (ppm)); 3.87 (3H, s), 7.30 (1H, dd, J=5.1, 2.9 Hz), 7.53 (1H, dd, J=5.1, 1.2 Hz), 8.11 (1H, dd, J=2.9, 1.2 Hz)

### Example 2

### [R=Methoxycarbonyl Group]; Synthesis of 3-methoxycarbonylthiophene

In a flask having an inner volume of 25 ml and equipped with a stirrer, a thermometer and a reflux condenser were charged 10 ml of 1,2-dichloroethane, 1.39 g (12.0 mmol) of methyl 3-methoxyacrylate and 0.76 g of 1,4-dithiane-2,5-diol (10 mmol as α-mercaptoacetaldehyde), then, 0.52 g (2 mmol) of tin tetrachloride was gradually added dropwise thereto while maintaining the liquid temperature to 20° C., and the mixture was reacted under stirring at 25° C. for 20 hours. After completion of the reaction, when the reaction mixture was analyzed by high performance liquid chromatography (absolute calibration method), it was found that 0.98 g (Reaction yield; 69%) of 3-methoxycarbonylthiophene was formed.

### Example 3

### [R=Methoxycarbonyl Group]; Synthesis of 3-methoxycarbonylthiophene

In a flask having an inner volume of 25 ml and equipped with a stirrer, a thermometer and a reflux condenser were charged 10 ml of 1,2-dichloroethane, 1.39 g (12.0 mmol) of methyl 3-methoxyacrylate, 0.76 g of 1,4-dithiane-2,5-diol (10 mmol as α-mercaptoacetaldehyde) and 0.54 g (2.0 mmol) of zinc chloride, and the mixture was reacted under stirring at 83° C. for 4 hours. After completion of the reaction, the reaction mixture was filtered, and when the filtrate was analyzed by high performance liquid chromatography (absolute calibration method), it was found that 0.61 g (Reaction yield; 43%) of 3-methoxycarbonylthiophene was formed.

### Example 4

### [R=Methoxycarbonyl Group]; Synthesis of 3-methoxycarbonylthiophene

In a flask having an inner volume of 25 ml and equipped with a stirrer, a thermometer and a reflux condenser were charged 10 ml of 1,2-dichloroethane, 1.39 g (12.0 mmol) of methyl 3-methoxyacrylate and 0.76 g of 1,4-dithiane-2,5-diol (10 mmol as α-mercaptoacetaldehyde), then, 1.42 g (10 mmol) of boron trifluoride-diethyl ether complex was gradually added dropwise thereto while maintaining the liquid temperature to 20° C., and the mixture was reacted under stirring at 60° C. for 4 hours. After completion of the reaction, when the reaction mixture was analyzed by high performance liquid chromatography (absolute calibration method), it was found that 0.55 g (Reaction yield; 39%) of 3-methoxycarbonylthiophene was formed.

### Example 5

### [R=Cyano Group]; Synthesis of 3-cyanothiophene

In a flask having an inner volume of 25 ml and equipped with a stirrer, a thermometer and a reflux condenser were charged 2.58 g (30.0 mmol) of 3-methoxypropenenitrile, 1.52 g of 1,4-dithiane-2,5-diol (20.0 mmol as α-mercaptoacetaldehyde), 2.76 g (20 mmol) of potassium carbonate and 20 ml of N,N-dimethylformamide, and the mixture was reacted under stirring at 100° C. for 16 hours. After completion of the reaction, the reaction mixture was filtered, and the filtrate was concentrated under reduced pressure. The concentrate was purified by silica gel column chromatography (Eluent; n-hexane/ethyl acetate=5/1 to 1/1 (volume ratio)) to obtain 0.91 g (Isolation yield; 42%) of 3-cyanothiophene as colorless powder.

Physical properties of the 3-cyanothiophene are as follows.

¹H-NMR (CDCl₃, δ (ppm)); 7.31 (1H, dd, J=5.1, 1.2 Hz), 7.43 (1H, dd, J=5.1, 2.9 Hz), 7.95 (1H, dd, J=2.9, 1.2 Hz)

### Example 6

### [R=Cyano Group]; Synthesis of 3-cyanothiophene

In a flask having an inner volume of 25 ml and equipped with a stirrer, a thermometer and a reflux condenser were charged 2.58 g (30.0 mmol) of 3-methoxypropenenitrile, 1.52 g of 1,4-dithiane-2,5-diol (20.0 mmol as α-mercaptoacetaldehyde) and 25 ml of N,N-dimethylformamide, then, while maintaining the liquid temperature to 60° C., 1.52 g (10 mmol) of 1,8-diazabicyclo[5,4,0]-7-undecene was added dropwise thereto at the same temperature, and the mixture was reacted under stirring at 100° C. for 16 hours. After completion of the reaction, when the reaction mixture was analyzed by high performance liquid chromatography (absolute calibration method), it was found that 1.31 g (Reaction yield; 60%) of 3-cyanothiophene was formed.

### Example 7

### [R=Cyano Group]; Synthesis of 3-cyanothiophene

In a flask having an inner volume of 25 ml and equipped with a stirrer, a thermometer and a reflux condenser were charged 2.58 g (30.0 mmol) of 3-methoxypropenenitrile, 1.52 g of 1,4-dithiane-2,5-diol (20.0 mmol as α-mercaptoacetaldehyde), 2.31 g (10 mmol) of rubidium carbonate and 20 ml of N,N-dimethylformamide, and the mixture was reacted under stirring at 100° C. for 6 hours. After completion of the reaction, the reaction mixture was filtered, and when the filtrate was analyzed by high performance liquid chromatography (absolute calibration method), it was found that 1.21 g (Reaction yield; 55%) of 3-cyanothiophene was formed.

### Example 8

### [R=Cyano Group]; Synthesis of 3-cyanothiophene

In a flask having an inner volume of 25 ml and equipped with a stirrer, a thermometer and a reflux condenser were charged 2.58 g (30.0 mmol) of 3-methoxypropenenitrile, 1.52 g of 1,4-dithiane-2,5-diol (20.0 mmol as α-mercaptoacetaldehyde), 1.63 g (5 mmol) of cesium carbonate and 20 ml of N,N-dimethylformamide, and the mixture was reacted under stirring at 100° C. for 6 hours. After completion of the reaction, the reaction mixture was filtered, and when the filtrate was analyzed by high performance liquid chromatography (absolute calibration method), it was found that 0.88 g (Reaction yield; 40%) of 3-cyanothiophene was formed.

### Example 9

### [R=Cyano Group]; Synthesis of 3-cyanothiophene

In a flask having an inner volume of 25 ml and equipped with a stirrer, a thermometer and a reflux condenser were charged 2.58 g (30.0 mmol) of 3-methoxypropenenitrile, 1.52 g of 1,4-dithiane-2,5-diol (20.0 mmol as α-mercaptoacetaldehyde), 0.56 g (5 mmol) of potassium t-butoxide and 20 ml of N,N-dimethylformamide, and the mixture was reacted under stirring at 25° C. for 67 hours. After completion of the reaction, the reaction mixture was filtered, and when the filtrate was analyzed by high performance liquid chromatography (absolute calibration method), it was found that 0.71 g (Reaction yield; 33%) of 3-cyanothiophene was formed.

### Example 10

### [R=Formyl Group]; Synthesis of 3-formylthiophene

In a flask having an inner volume of 25 ml and equipped with a stirrer, a thermometer and a reflux condenser were charged 1.72 g (20.0 mmol) of 3-methoxyacrolein, 1.52 g of 1,4-dithiane-2,5-diol (20.0 mmol as α-mercaptoacetaldehyde) and 10 ml of acetonitrile, then, 1.52 g (10 mmol) of 1,8-diazabicyclo[5,4,0]-7-undecene was gradually added dropwise thereto while maintaining the liquid temperature to 10° C., and the mixture was reacted under stirring at 10° C. for 2 hours. After completion of the reaction, the reaction mixture was distilled under reduced pressure (60 to 61° C., 2.4×10⁻³ MPa) to obtain 0.51 g (Isolation yield; 23%) of 3-formylthiophene as colorless oily product.

Physical properties of the 3-formylthiophene are as follows.

¹H-NMR (CDCl₃, δ (ppm)); 7.38 (1H, ddd, J=5.1, 2.9, 0.7 Hz), 7.54 (1H, dd, J=5.1, 1.2 Hz), 8.13 (1H, dd, J=2.9, 1.2 Hz), 9.93 (1H, d, 0.7 Hz)

### Reference Example 1

### [R=Acetyl Group, Y=Dimethylamino Group]; Synthesis of 1-(N,N-dimethylamino)-1-buten-3-one

In a flask having an inner volume of 300 ml and equipped with a stirrer, a thermometer and a reflux condenser were charged 142.7 g (1.08 mol) of 4,4-dimethoxy-2-butanone and 106.7 g (1.18 mmol) of a 50% by weight dimethylamine aqueous solution, and the mixture was reacted under stirring at 25° C. for 4 hours. After completion of the reaction, the reaction mixture was concentrated, and the concentrate was distilled under reduced pressure (115 to 120° C., 900 Pa) to obtain 117.1 g (Isolation yield; 95%) of 1-(N,N-dimethylamino)-1-buten-3-one as pale yellowish liquid.

Physical properties of the 1-(N,N-dimethylamino)-1-buten-3-one are as follows.

¹H-NMR (CDCl₃, δ (ppm)); 2.10 (3H, s), 2.94 (6H, brs), 5.05 (1H, d, J=12.7 Hz), 7.47 (1H, d, J=12.7 Hz)

### Example 11

### [R=Acetyl Group]; Synthesis of 3-acetylthiophene

In a flask having an inner volume of 300 ml and equipped with a stirrer, a thermometer and a reflux condenser were charged 1.13 g (10.0 mmol) of 1-(N,N-di-methylamino)-1-buten-3-one synthesized in the same manner as in Reference example 1 and 1.14 g of 1,4-dithiane-2,5-diol (14.8 mmol as α-mercaptoacetaldehyde), and the mixture was reacted under stirring at 110° C. for 30 hours. After completion of the reaction, the reaction mixture was concentrated and the concentrate was purified by silica gel column chromatography (Eluent; n-hexane/ethyl acetate=5/1 to 1/1 (volume ratio)) to obtain 0.11 g (Isolation yield; 96) of 3-acetylthiophene as colorless powder.

Physical properties of the 3-acetylthiophene are as follows.

¹H-NMR (CDCl₃, δ (ppm)); 2.54 (3H, s), 7.32 (1H, dd, J=5.0, 2.9 Hz), 7.54 (1H, dd, J=5.0, 1.2 Hz), 8.04 (1H, dd, J=2.9, 1.2 Hz)

### Example 12

### [R=Acetyl Group]; Synthesis of 3-Acetylthiophene

In a flask having an inner volume of 50 ml and equipped with a stirrer, a thermometer and a reflux condenser were charged 2.26 g (20.0 mmol) of 1-(N,N-dimethylamino)-1-buten-3-one synthesized in the same manner as in Reference example 1, 1.52 g of 1,4-dithiane-2,5-diol (20.0 mmol as α-mercaptoacetaldehyde), 0.15 g (1.0 mmol) of 1,8-diazabicyclo[5,4,0]-7-undecene and 20 ml of N,N-dimethylformamide, and the mixture was reacted under stirring at 67° C. for 10 hours. After completion of the reaction, when the reaction mixture was analyzed by high performance liquid chromatography (absolute calibration method), it was found that 0.41 g (Reaction yield; 16%) of 3-acetylthiophene was formed.

### Example 13

### [R=Acetyl Group]; Synthesis of 3-acetylthiophene

In a flask having an inner volume of 50 ml and equipped with a stirrer, a thermometer and a reflux condenser were charged 2.26 g (20.0 mmol) of 1-(N,N-dimethylamino)-1-buten-3-one synthesized in the same manner as in Reference example 1, 1.52 g of 1,4-dithiane-2,5-diol (20.0 mmol as α-mercaptoacetaldehyde), 0.28 g (2.0 mmol) of potassium carbonate and 20 ml of N,N-dimethylformamide, and the mixture was reacted under stirring at 80° C. for 8 hours. After completion of the reaction, when the reaction mixture was analyzed by high performance liquid chromatography (absolute calibration method), it was found that 0.43 g (Reaction yield; 17%) of 3-acetylthiophene was formed.

### Example 14

### [R=Acetyl Group]; Synthesis of 3-acetylthiophene

In a flask having an inner volume of 50 ml and equipped with a stirrer, a thermometer and a reflux condenser were charged 1.13 g (10.0 mmol) of 1-(N,N-dimethylamino)-1-buten-3-one synthesized in the same manner as in Reference example 1, 1.16 g of 1,4-dithiane-2,5-diol (15.0 mmol as α-mercaptoacetaldehyde), 0.51 g (5.0 mmol) of conc. sulfuric acid and 15 ml of n-butyl alcohol, and the mixture was reacted under stirring at 90° C. for 10 hours. After completion of the reaction, when the reaction mixture was analyzed by high performance liquid chromatography (absolute calibration method), it was found that 0.42 g (Reaction yield; 33%) of 3-acetylthiophene was formed.

### Example 15

### [R=Acetyl Group]; Synthesis of 3-acetylthiophene

In a flask having an inner volume of 50 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.76 g of 1,4-dithiane-2,5-diol (10.0 mmol as α-mercaptoacetaldehyde), 0.95 g (5.0 mmol) of p-toluenesulfonic acid monohydrate and 10 ml of toluene, and the mixture was reacted under stirring at 90° C. for 3 hours. After completion of the reaction, when the reaction mixture was analyzed by high performance liquid chromatography (absolute calibration method), it was found that 0.35 g (Reaction yield; 28%) of 3-acetylthiophene was formed.

### Example 16

### [R=Acetyl Group]; Synthesis of 3-acetylthiophene

In a flask having an inner volume of 50 ml and equipped with a stirrer, a thermometer and a reflux condenser were charged 1.13 g (10.0 mmol) of 1-(N,N-dimethylamino)-1-buten-3-one synthesized in the same manner as in Reference example 1, 1.16 g of 1,4-dithiane-2,5-diol (15.0 mmol as α-mercaptoacetaldehyde), 0.50 g (5.0 mmol) of conc. hydrochloric acid and 10 ml of toluene, and the mixture was reacted under stirring at 90° C. for 10 hours. After completion of the reaction, when the reaction mixture was analyzed by high performance liquid chromatography (absolute calibration method), it was found that 0.40 g (Reaction yield; 32%) of 3-acetylthiophene was formed.

### Example 17

### [R=Acetyl Group]; Synthesis of 3-acetylthiophene

In a flask having an inner volume of 50 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.76 g of 1,4-dithiane-2,5-diol (10.0 mmol as α-mercaptoacetaldehyde), 0.54 g (4.0 mmol) of zinc chloride and 10 ml of propionitrile, and the mixture was reacted under stirring at 97° C. for 17 hours. After completion of the reaction, when the reaction mixture was analyzed by high performance liquid chromatography (absolute calibration method), it was found that 0.31 g (Reaction yield; 25%) of 3-acetylthiophene was formed.

### Example 18

### [R=Acetyl Group]; Synthesis of 3-acetylthiophene

In a flask having an inner volume of 50 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.76 g of 1,4-dithiane-2,5-diol (10.0 mmol as α-mercaptoacetaldehyde), 0.48 g (5.0 mmol) of magnesium chloride and 10 ml of propionitrile, and the mixture was reacted under stirring at 97° C. for 17 hours. After completion of the reaction, when the reaction mixture was analyzed by high performance liquid chromatography (absolute calibration method), it was found that 0.29 g (Reaction yield; 23%) of 3-acetylthiophene was formed.

### Example 19

### [R=Acetyl Group]; Synthesis of 3-acetylthiophene

In a flask having an inner volume of 25 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.76 g of 1,4-dithiane-2,5-diol (10.0 mmol as α-mercaptoacetaldehyde), 0.57 g (2.0 mmol) of titanium tetraisopropoxide and 10 ml of 1,2-dichloroethane, and the mixture was reacted under stirring at 82° C. for 2 hours. After completion of the reaction, when the reaction mixture was analyzed by high performance liquid chromatography (absolute calibration method), it was found that 0.25 g (Reaction yield; 20%) of 3-acetylthiophene was formed.

### Example 20

### [R=Acetyl Group]; Synthesis of 3-acetylthiophene

In a flask having an inner volume of 25 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.76 g of 1,4-dithiane-2,5-diol (10.0 mmol as α-mercaptoacetaldehyde), 0.38 g (2.0 mmol) of titanium tetrachloride and 10 ml of 1,2-dichloroethane, and the mixture was reacted under stirring at 82° C. for 2 hours. After completion of the reaction, when the reaction mixture was analyzed by high performance liquid chromatography (absolute calibration method), it was found that 0.68 g (Reaction yield; 54%) of 3-acetylthiophene was formed.

### Example 21

### [R=Acetyl Group]; Synthesis of 3-acetylthiophene

In a flask having an inner volume of 25 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.76 g of 1,4-dithiane-2,5-diol (10.0 mmol as α-mercaptoacetaldehyde), 0.38 g (2.0 mmol) of titanium tetrachloride and 10 ml of toluene, and the mixture was reacted under stirring at 82° C. for 2 hours. After completion of the reaction, when the reaction mixture was analyzed by high performance liquid chromatography (absolute calibration method), it was found that 0.58 g (Reaction yield; 46%) of 3-acetylthiophene was formed.

### Example 22

### [R=Acetyl Group]; Synthesis of 3-acetylthiophene

In a flask having an inner volume of 25 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.76 g of 1,4-dithiane-2,5-diol (10.0 mmol as α-mercaptoacetaldehyde), 0.38 g (2.0 mmol) of titanium tetrachloride and 10 ml of chlorobenzene, and the mixture was reacted under stirring at 82° C. for 2 hours. After completion of the reaction, when the reaction mixture was analyzed by high performance liquid chromatography (absolute calibration method), it was found that 0.59 g (Reaction yield; 47%) of 3-acetylthiophene was formed.

### Example 23

### [R=Acetyl Group]; Synthesis of 3-acetylthiophene

In a flask having an inner volume of 25 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.76 g of 1,4-dithiane-2,5-diol (10.0 mmol as α-mercaptoacetaldehyde), 0.38 g (2.0 mmol) of titanium tetrachloride and 10 ml of tetrahydrofuran, and the mixture was reacted under stirring at 67° C. for 2 hours. After completion of the reaction, when the reaction mixture was analyzed by high performance liquid chromatography (absolute calibration method), it was found that 0.63 g (Reaction yield; 50%) of 3-acetylthiophene was formed.

### Example 24

### [R=Acetyl Group]; Synthesis of 3-acetylthiophene

In a flask having an inner volume of 25 ml and equipped with a stirrer, a thermometer and a reflux condenser were charged 3.40 g (30.0 mmol) of 1-(N,N-dimethylamino)-1-buten-3-one synthesized in the same manner as in Reference example 1, 2.78 g of 1,4-dithiane-2,5-diol (36.0 mmol as α-mercaptoacetaldehyde), 1.14 g (6.0 mmol) of titanium tetrachloride and 54 ml of 1,2-dichloroethane, and the mixture was reacted under stirring at 80° C. for 6 hours. After completion of the reaction, when the reaction mixture was analyzed by high performance liquid chromatography (absolute calibration method), it was found that 0.76 g (Reaction yield; 60%) of 3-acetylthiophene was formed.

### Example 25

### [R=Acetyl Group]; Synthesis of 3-acetylthiophene

In a flask having an inner volume of 25 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.76 g of 1,4-dithiane-2,5-diol (10.0 mmol as α-mercaptoacetaldehyde), 0.52 g (2.0 mmol) of tin tetrachloride and 10 ml of 1,2-dichloroethane, and the mixture was reacted under stirring at 80° C. for 2 hours. After completion of the reaction, when the reaction mixture was analyzed by high performance liquid chromatography (absolute calibration method), it was found that 0.48 g (Reaction yield; 38%) of 3-acetylthiophene was formed.

### Example 26

### [R=Acetyl Group]; Synthesis of 3-acetylthiophene)

In a flask having an inner volume of 25 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.76 g of 1,4-dithiane-2,5-diol (10.0 mmol as α-mercaptoacetaldehyde), 1.42 g (10.0 mmol) of boron trifluoride-diethyl ether complex and 10 ml of 1,2-dichloroethane, and the mixture was reacted under stirring at 67° C. for 7 hours. After completion of the reaction, when the reaction mixture was analyzed by high performance liquid chromatography (absolute calibration method, it was found that 0.65 g (Reaction yield; 52%) of 3-acetylthiophene was formed.

### Example 27

### [R=Acetyl Group]; Synthesis of 3-acetylthiophene

In a flask having an inner volume of 25 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.76 g of 1,4-dithiane-2,5-diol (10.0 mmol as α-mercaptoacetaldehyde), 1.42 g (10.0 mmol) of boron trifluoride.diethyl ether complex and 10 ml of tetrahydrofuran, and the mixture was reacted under stirring at 67° C. for 7 hours. After completion of the reaction, when the reaction mixture was analyzed by high performance liquid chromatography (absolute calibration method, it was found that 0.64 g (Reaction yield; 51%) of 3-acetylthiophene was formed.

### Example 28

### [R=Acetyl Group]; Synthesis of 3-acetylthiophene

In a flask having an inner volume of 25 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.76 g of 1,4-dithiane-2,5-diol (10.0 mmol as α-mercaptoacetaldehyde), 1.42 g (10.0 mmol) of boron trifluoride.diethyl ether complex and 10 ml of toluene, and the mixture was reacted under stirring at 67° C. for 7 hours. After completion of the reaction, when the reaction mixture was analyzed by high performance liquid chromatography (absolute calibration method, it was found that 0.55 g (Reaction yield; 44%) of 3-acetylthiophene was formed.

### Example 29

### [R=Acetyl Group]; Synthesis of 3-acetylthiophene

In a flask having an inner volume of 25 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.76 g of 1,4-dithiane-2,5-diol (10.0 mmol as α-mercaptoacetaldehyde), 1.42 g (10.0 mmol) of boron trifluoride-diethyl ether complex and 10 ml of acetonitrile, and the mixture was reacted under stirring at 67° C. for 7 hours. After completion of the reaction, when the reaction mixture was analyzed by high performance liquid chromatography (absolute calibration method, it was found that 0.60 g (Reaction yield; 48%) of 3-acetylthiophene was formed.

### Example 30

### [R=Acetyl Group]; Synthesis of 3-acetylthiophene

In a flask having an inner volume of 25 ml and equipped with a stirrer, a thermometer and a reflux condenser were charged 1.13 g (10.0 mmol) of 1-(N,N-dimethylamino)-1-buten-3-one synthesized in the same manner as in Reference example 1, 0.93 g of 1,4-dithiane-2,5-diol (12.0 mmol as α-mercaptoacetaldehyde), 1.42 g (10.0 mmol) of boron trifluoride-diethyl ether complex and 10 ml of 1,2-dichloroethane, and the mixture was reacted under stirring at 67° C. for 7 hours. After completion of the reaction, when the reaction mixture was analyzed by high performance liquid chromatography (absolute calibration method, it was found that 0.71 g (Reaction yield; 56%) of 3-acetylthiophene was formed.

### Reference Example 2

### [R=Benzoyl Group, Y=Dimethylamino Group]; Synthesis of 3-(N,N-dimethylamino)-1-phenyl-2-propenone

In a flask having an inner volume of 50 ml and equipped with a stirrer, a thermometer and a reflux condenser were charged 9.62 g (80 mmol) of acetophenone and 10.5 g (88 mmol) of N,N-dimethylformamide dimethylacetal, and the mixture was reacted under stirring at 88 to 90° C. for 30 hours. After completion of the reaction, the reaction mixture was concentrated, and 8 ml of n-butyl alcohol was added to the concentrate to precipitate crystals to obtain 11.1 g (Isolation yield; 79%) of 3-(N,N-dimethylamino)-1-phenyl-2-propenone as yellowish needle crystals.

Physical properties of the 3-(N,N-dimethylamino)-1-phenyl-2-propenone are as follows.

¹H-NMR (CDCl₃, δ (ppm)); 2.94 (6H, brs), 5.72 (1H, d, J=12.5 Hz), 7.35 to 7.50 (3H, m), 7.80 (1H, d, J=12.5 Hz), 7.85 to 7.93 (2H, m)

### Example 31

### [R=Benzoyl Group]; Synthesis of 3-benzoylthiophene

In a flask having an inner volume of 25 ml and equipped with a stirrer, a thermometer and a reflux condenser were charged 1.75 g (10.0 mmol) of 3-(N,N-dimethylamino)-1-phenyl-2-propenone synthesized in the same manner as in Reference example 2, 0.76 g of 1,4-dithiane-2,5-diol (10.0 mmol as α-mercaptoacetaldehyde), 0.38 g (2.0 mmol) of titanium tetrachloride and 10 ml of 1,2-dichloroethane, and the mixture was reacted under stirring at 67° C. for 7 hours. After completion of the reaction, the reaction mixture was concentrated and the concentrate was purified by silica gel column chromatography (Eluent; n-hexane/ethyl acetate=3/1 to 1/1 (volume ratio)) to obtain 1.10 g (Isolation yield; 58%) of 3-benzoylthiophene as pale yellowish liquid.

Physical properties of the 3-benzoylthiophene are as follows.

¹H-NMR (CDCl₃, δ (ppm)); 7.38 (1H, dd, J=5.1, 2.9 Hz), 7.45 to 7.59 (3H, m), 7.61 (1H, dd, J=5.1, 1.2 Hz), 7.80 to 7.89 (2H, m), 7.93 (1H, dd, J=2.9, 1.2 Hz)

### Example 32

### [R=Benzoyl Group]; Synthesis of 3-benzoylthiophene

In a flask having an inner volume of 25 ml and equipped with a stirrer, a thermometer and a reflux condenser were charged 3.50 g (20.0 mmol) of 3-(N,N-dimethylamino)-1-phenyl-2-propenone synthesized in the same manner as in Reference example 2, 1.86 g of 1,4-dithiane-2,5-diol (24.0 mmol as α-mercaptoacetaldehyde), 0.76 g (4.0 mmol) of titanium tetrachloride and 20 ml of 1,2-dichloroethane, and the mixture was reacted under stirring at 80° C. for 5 hours. After completion of the reaction, when the reaction mixture was analyzed by high performance liquid chromatography (absolute calibration method), it was found that 2.50 g (Reaction yield; 67%) of 3-benzoylthiophene was formed.

### Reference Example 3

### [R=n-valeryl Group, Y=dimethylamino Group]; Synthesis of 1-(N,N-dimethylamino)-1-hepten-3-one

In a flask having an inner volume of 300 ml and equipped with a stirrer, a thermometer and a reflux condenser were charged 100.2 g (1.0 mmol) of 2-hexanone and 59.6 g (0.50 mol) of N,N-dimethylformamide dimethylacetal, and the mixture was reacted under stirring at 115 to 120° C. for 10 hours. After completion of the reaction, the reaction mixture was concentrated and the concentrate was purified by silica gel column chromatography (Eluent; ethyl acetate) to obtain 45.0 g (Isolation yield; 58%) of 1-(N,N-dimethylamino)-1-hepten-3-one as pale brownish liquid.

Physical properties of the 1-(N,N-dimethylamino)-1-hepten-3-one are as follows.

¹H-NMR (CDCl₃, δ (ppm)); 0.91 (1H, t, J=7.3 Hz), 1.30 to 1.41 (2H, m), 1.53 to 1.70 (2H, m), 2.25 to 2.37 (2H, m), 2.93 (6H, brs), 5.40 (1H, d, J=12.7 Hz), 7.51 (1H, d, J=12.7 Hz)

### Example 33

### [R=n-valeryl Group]; Synthesis of 3-valerylthiophene

In a flask having an inner volume of 25 ml and equipped with a stirrer, a thermometer and a reflux condenser were charged 3.11 g (20.0 mmol) of 1-(N,N-dimethylamino)-1-hepten-3-one synthesized in the same manner as in Reference example 3, 1.85 g of 1,4-dithiane-2,5-diol (24.0 mmol as α-mercaptoacetaldehyde), 0.76 g (2.0 mmol) of titanium tetrachloride and 36 ml of 1,2-dichloroethane, and the mixture was reacted under stirring at 82° C. for 10 hours. After completion of the reaction, the reaction mixture was concentrated and the concentrate was purified by silica gel column chromatography (Eluent; n-hexane/ethyl acetate=3/1 to 1/1 (volume ratio)) to obtain 2.10 g (Isolation yield; 63%) of 3-valerylthiophene as pale yellowish liquid.

Physical properties of the 3-valerylthiophene are as follows.

¹H-NMR (CDCl₃, δ (ppm)); 0.95 (3H, t, J=7.3 Hz), 1.38 to 1.45 (2H, m), 1.67 to 1.80 (2H, m), 2.83 to 2.90 (2H, m), 7.31 (1H, dd, J=5.0, 2.8 Hz), 7.55 (1H, dd, J=5.0, 1.2 Hz), 8.04 (1H, dd, J=2.8, 1.2 Hz)

### Reference Example 4

### [R=n-octanoyl Group, Y=Dimethylamino Group]; Synthesis of 1-(N,N-dimethylamino)-1-decen-3-one

In a flask having an inner volume of 300 ml and equipped with a stirrer, a thermometer and a reflux condenser were charged 14.2 g (0.10 mol) of 2-nonanone, 5.96 g (0.05 mol) of N,N-dimethylformamide dimethylacetal and 36 ml of N,N-dimethylformamide, and the mixture was reacted under stirring at 110 to 120° C. for 10 hours. After completion of the reaction, the reaction mixture was concentrated and the concentrate was purified by silica gel column chromatography (Eluent; ethyl acetate) to obtain 5.73 g (Isolation yield; 58%) of 1-(N,N-dimethylamino)-1-decen-3-one as pale brownish liquid.

Physical properties of the 1-(N,N-dimethylamino)-1-decen-3-one are as follows.

¹H-NMR (CDCl₃, δ (ppm)); 0.87 (1H, t, J=6.8 Hz), 1.20 to 1.50 (8H, m), 1.50 to 1.70 (2H, m), 2.28 to 2.38 (2H, m), 2.90 (6H, brs), 5.04 (1H, d, J=12.7 Hz), 7.53 (1H, d, J=12.7 Hz)

### Example 34

### [R=n-octanoyl Group]; Synthesis of 3-octanoylthiophene

In a flask having an inner volume of 25 ml and equipped with a stirrer, a thermometer and a reflux condenser were charged 1.86 g (9.43 mmol) of 1-(N,N-dimethylamino)-1-decen-3-one synthesized in the same manner as in Reference example 4, 0.87 g of 1,4-dithiane-2,5-diol (11.3 mmol as α-mercaptoacetaldehyde), 0.36 g (1.9 mmol) of titanium tetrachloride and 15 ml of 1,2-dichloroethane, and the mixture was reacted under stirring at 82° C. for 4 hours. After completion of the reaction, the reaction mixture was concentrated and the concentrate was purified by silica gel column chromatography (Eluent; n-hexane/ethyl acetate=8/1 (volume ratio)) to obtain 1.10 g (Isolation yield; 56%) of 3-octanoylthiophene as pale yellowish liquid.

Physical properties of the 3-octanoylthiophene are as follows.

¹H-NMR (CDCl₃, δ (ppm)); 0.88 (3H, t, J=6.8 Hz), 1.25 to 1.40 (8H, m), 1.60 to 1.80 (2H, m), 2.82 to 2.90 (2H, m), 7.31 (1H, dd, J=5.1, 2.9 Hz), 7.54 (1H, dd, J=5.1, 1.2 Hz), 8.04 (1H, dd, J=2.9, 1.2 Hz)

### Example 35

### [R=Acetyl Group]; Synthesis of 3-acetylthiophene

In a flask having an inner volume of 25 ml and equipped with a stirrer, a thermometer and a reflux condenser were charged 1.00 g (10.0 mmol) of 4-methoxy-3-buten-2-one and 1.14 g of 1,4-dithiane-2,5-diol (14.8 mmol as α-mercaptoacetaldehyde), and the mixture was reacted under stirring at 110° C. for 30 hours. After completion of the reaction, the reaction mixture was concentrated and the concentrate was purified by silica gel column chromatography (Eluent; n-hexane/ethyl acetate=5/1 to 1/1 (volume ratio)) to obtain 0.26 g (Isolation yield; 21%) of 3-acetylthiophene as colorless powder.

Physical properties of the 3-acetylthiophene are as follows.

¹H-NMR (CDCl₃, δ (ppm)); 2.54 (3H, s), 7.32 (1H, dd, J=5.0, 2.9 Hz), 7.54 (1H, dd, J=5.0, 1.2 Hz), 8.04 (1H, dd, J=2.9, 1.2 Hz)

### Example 36

### [R=Acetyl Group]; Synthesis of 3-acetylthiophene

In a flask having an inner volume of 50 ml and equipped with a stirrer, a thermometer and a reflux condenser were charged 2.00 g (20.0 mmol) of 4-methoxy-3-buten-2-one, 1.52 g of 1,4-dithiane-2,5-diol (20.0 mmol as α-mercaptoacetaldehyde), 0.15 g (1.0 mmol) of 1,8-diazabicyclo[5,4,0]-7-undecene and 20 ml of acetonitrile, and the mixture was reacted under stirring at 67° C. for 3 hours. Then, 0.4 g (4.0 mmol) of conc. hydrochloric acid and 5 ml of water were added to the mixture, and the mixture was further reacted under stirring at 60° C. for 3.5 hours. After completion of the reaction, when the reaction mixture was analyzed by high performance liquid chromatography (absolute calibration method), it was found that 2.35 g (Reaction yield; 93%) of 3-acetylthiophene was formed.

### Example 37

### [R=Acetyl Group]; Synthesis of 3-acetylthiophene

In a flask having an inner volume of 50 ml and equipped with a stirrer, a thermometer and a reflux condenser were charged 3.00 g (30.0 mmol) of 4-methoxy-3-buten-2-one, 1.52 g of 1,4-dithiane-2,5-diol (20.0 mmol as α-mercaptoacetaldehyde), 0.28 g (2.0 mmol) of potassium carbonate and 20 ml of propionitrile, and the mixture was reacted under stirring at 97° C. for 8 hours, then, 0.4 g (4.0 mmol) of conc. hydrochloric acid and 5 ml of water were added to the mixture, and the mixture was further reacted under stirring at 60° C. for 4 hours. After completion of the reaction, when the reaction mixture was analyzed by high performance liquid chromatography (absolute calibration method), it was found that 2.25 g (Reaction yield; 89%) of 3-acetylthiophene was formed.

### Example 38

### [R=Acetyl Group]; Synthesis of 3-acetylthiophene

In a flask having an inner volume of 50 ml and equipped with a stirrer, a thermometer and a reflux condenser were charged 3.00 g (30.0 mmol) of 4-methoxy-3-buten-2-one, 1.52 g of 1,4-dithiane-2,5-diol (20.0 mmol as α-mercaptoacetaldehyde), 0.28 g (2.0 mmol) of potassium carbonate and 20 ml of N,N-dimethylformamide, and the mixture was reacted under stirring at 97° C. for 8 hours, then, 0.4 g (4.0 mmol) of conc. hydrochloric acid and 5 ml of water were added to the mixture, and the mixture was further reacted under stirring at 60° C. for 4 hours. After completion of the reaction, 10 ml of ethyl acetate was added to the reaction mixture, when it was analyzed by high performance liquid chromatography (absolute calibration method), it was found that 1.95 g (Reaction yield; 77%) of 3-acetylthiophene was formed.

### Example 39

### [R=Acetyl Group]; Synthesis of 3-acetylthiophene

In a flask having an inner volume of 50 ml and equipped with a stirrer, a thermometer and a reflux condenser were charged 3.00 g (30.0 mmol) of 4-methoxy-3-buten-2-one, 1.52 g of 1,4-dithiane-2,5-diol (20.0 mmol as α-mercaptoacetaldehyde), 1.52 g (10 mmol) of 1,8-diazabicyclo[5,4,0]-7-undecene and 20 ml of tetrahydrofuran, and the mixture was reacted under stirring at 67° C. for 8 hours. After completion of the reaction, when the reaction mixture was analyzed by high performance liquid chromatography (absolute calibration method), it was found that 0.71 g (Reaction yield; 28%) of 3-acetylthiophene was formed.

### Example 40

### [R=Acetyl Group]; Synthesis of 3-acetylthiophene

In a flask having an inner volume of 25 ml and equipped with a stirrer, a thermometer and a reflux condenser were charged 1.00 g (10.0 mmol) of 4-methoxy-3-buten-2-one, 1.14 g of 1,4-dithiane-2,5-diol (14.8 mmol as α-mercaptoacetaldehyde) and 60 ml of acetonitrile, then, the liquid temperature was raised to up to 50° C., 0.76 g (5 mmol) of 1,8-diazabicyclo[5,4,0]-7-undecene was gradually added to the mixture, and the mixture was reacted under stirring at 60° C. for 8 hours. Then, 1.04 g (10 mmol) of conc. hydrochloric acid and 40 ml of water were added to the mixture, and the mixture was further reacted under stirring at 60° C. for 2.5 hours. After completion of the reaction, the reaction mixture was concentrated and the concentrate was distilled under reduced pressure (82° C., 1.07 to 1.2 kPa) to obtain 10.8 g (Isolation yield; 86%) of 3-acetylthiophene as colorless powder. The obtained powder was recrystallized from 16 ml of n-hexane/1.5 ml of toluene to obtain 9.93 g (Isolation yield; 79%) of higher purity 3-acetylthiophene as colorless powder.

### Example 41

### [R=Acetyl Group]; Synthesis of 3-acetylthiophene

In a flask having an inner volume of 50 ml and equipped with a stirrer, a thermometer and a reflux condenser were charged 4.20 g (42.0 mmol) of 4-methoxy-3-buten-2-one, 2.13 g of 1,4-dithiane-2,5-diol (28.0 mmol as α-mercaptoacetaldehyde) and 28 ml of acetonitrile, the liquid temperature was raised to up to 50° C., 0.021 g (0.14 mmol) of 1,8-diazabicyclo[5,4,0]-7-undecene was gradually added to the mixture, and the mixture was reacted under stirring at 67° C. for 10 hours. Then, 0.10 g (1.0 mmol) of conc. hydrochloric acid and 1 ml of water were added to the mixture, and the mixture was further reacted under stirring at 60° C. for 2.5 hours. After completion of the reaction, 20 ml of ethyl acetate was added to the reaction mixture, and when it was analyzed by high performance liquid chromatography (absolute calibration method), it was found that 2.54 g (Reaction yield; 72%) of 3-acetylthiophene was formed.

### Example 42

### [R=Acetyl Group]; Synthesis of 3-acetylthiophene

In a flask having an inner volume of 50 ml and equipped with a stirrer, a thermomether and a reflux condenser were charged 4.20 g (42.0 mmol) of 4-methoxy-3-buten-2-one, 2.13 g of 1,4-dithiane-2,5-diol (28.0 mmol as α-mercaptoacetaldehyde) and 28 ml of N,N-dimethylformamide, the liquid temperature was raised to up to 50° C., 0.021 g (0.14 mmol) of 1,8-diazabicyclo[5,4,0]-7-undecene was gradually added to the mixture, and the mixture was reacted under stirring at 67° C. for 10 hours. Then, 0.10 g (1.0 mmol) of conc. hydrochloric acid and 1 ml of water were added to the mixture, and the mixture was further reacted under stirring at 60° C. for 2.5 hours. After completion of the reaction, 20 ml of ethyl acetate was added to the reaction mixture, and when it was analyzed by high performance liquid chromatography (absolute calibration method), it was found that 2.14 g (Reaction yield; 61%) of 3-acetylthiophene was formed.

### Example 43

### [R=Acetyl Group]; Synthesis of 3-acetylthiophene

In a flask having an inner volume of 50 ml and equipped with a stirrer, a thermomether and a reflux condenser were charged 4.20 g (42.0 mmol) of 4-methoxy-3-buten-2-one, 2.13 g of 1,4-dithiane-2,5-diol (28.0 mmol as α-mercaptoacetaldehyde) and 28 ml of t-butyl alcohol, the liquid temperature was raised to up to 50° C., 0.021 g (0.14 mmol) of 1,8-diazabicyclo[5,4,0]-7-undecene was gradually added to the mixture, and the mixture was reacted under stirring at 67° C. for 10 hours. Then, 0.10 g (1.0 mmol) of conc. hydrochloric acid and 1 ml of water were added to the mixture, and the mixture was further reacted under stirring at 60° C. for 2.5 hours. After completion of the reaction, 20 ml of ethyl acetate was added to the reaction mixture, and when it was analyzed by high performance liquid chromatography (absolute calibration method), it was found that 2.25 g (Reaction yield; 64%) of 3-acetylthiophene was formed.

### Example 44

### [R=Acetyl Group]; Synthesis of 3-acetylthiophene

In a flask having an inner volume of 25 ml and equipped with a stirrer, a thermomether and a reflux condenser were charged 3.00 g (30.0 mmol) of 4-methoxy-3-buten-2-one, 1.54 g of 1,4-dithiane-2,5-diol (20.0 mmol as α-mercaptoacetaldehyde), 0.19 g (1.0 mmol) of p-toluenesulfonic acid monohydrate and 10 ml of toluene, and the mixture was reacted under stirring at 67° C. for 5 hours. After completion of the reaction, when the reaction mixture was analyzed by high performance liquid chromatography (absolute calibration method), it was found that 0.75 g (Reaction yield; 59%) of 3-acetylthiophene was formed.

### Example 45

### [R=Acetyl Group]; Synthesis of 3-acetylthiophene

In a flask having an inner volume of 25 ml and equipped with a stirrer, a thermomether and a reflux condenser were charged 1.00 g (10.0 mmol) of 4-methoxy-3-buten-2-one, 0.76 g of 1,4-dithiane-2,5-diol (10.0 mmol as α-mercaptoacetaldehyde), 0.49 g (5.0 mmol) of conc. hydrochloric acid and 10 ml of toluene, and the mixture was reacted under stirring at 65° C. for 3 hours. After completion of the reaction, when the reaction mixture was analyzed by high performance liquid chromatography (absolute calibration method), it was found that 0.25 g (Reaction yield; 20%) of 3-acetylthiophene was formed.

### Example 46

### [R=Acetyl Group]; Synthesis of 3-acetylthiophene

In a flask having an inner volume of 25 ml and equipped with a stirrer, a thermomether and a reflux condenser were charged 1.00 g (10.0 mmol) of 4-methoxy-3-buten-2-one, 0.76 g of 1,4-dithiane-2,5-diol (10.0 mmol as α-mercaptoacetaldehyde), 0.19 g (1.0 mmol) of titanium tetrachloride and 10 ml of toluene, and the mixture was reacted under stirring at 85° C. for 30 minutes. After completion of the reaction, when the reaction mixture was analyzed by high performance liquid chromatography (absolute calibration method), it was found that 0.67 g (Reaction yield; 53%) of 3-acetylthiophene was formed.

### Example 47

### [R=Acetyl Group]; Synthesis of 3-acetylthiophene

In a flask having an inner volume of 25 ml and equipped with a stirrer, a thermomether and a reflux condenser were charged 1.00 g (10.0 mmol) of 4-methoxy-3-buten-2-one, 0.76 g of 1,4-dithiane-2,5-diol (10.0 mmol as α-mercaptoacetaldehyde), 0.19 g (1.0 mmol) of titanium tetrachloride and 10 ml of 1,2-dichloroethane, and the mixture was reacted under stirring at 85° C. for 30 minutes. After completion of the reaction, when the reaction mixture was analyzed by high performance liquid chromatography (absolute calibration method), it was found that 0.87 g (Reaction yield; 69%) of 3-acetylthiophene was formed.

### Example 48

### [R=Acetyl Group]; Synthesis of 3-acetylthiophene

In a flask having an inner volume of 25 ml and equipped with a stirrer, a thermomether and a reflux condenser were charged 1.00 g (10.0 mmol) of 4-methoxy-3-buten-2-one, 0.76 g of 1,4-dithiane-2,5-diol (10.0 mmol as α-mercaptoacetaldehyde), 0.27 g (1.0 mmol) of zinc chloride and 10 ml of propionitrile, and the mixture was reacted under stirring at 97° C. for 3 hours. After completion of the reaction, when the reaction mixture was analyzed by high performance liquid chromatography (absolute calibration method), it was found that 0.61 g (Reaction yield; 48%) of 3-acetylthiophene was formed.

### Example 49

### [R=Acetyl Group]; Synthesis of 3-acetylthiophene

In a flask having an inner volume of 25 ml and equipped with a stirrer, a thermomether and a reflux condenser were charged 1.00 g (10.0 mmol) of 4-methoxy-3-buten-2-one, 0.76 g of 1,4-dithiane-2,5-diol (10.0 mmol as α-mercaptoacetaldehyde), 0.57 g (2.0 mmol) of titanium tetraisopropoxide and 10 ml of propionitrile, and the mixture was reacted under stirring at 90° C. for 5 hours. After completion of the reaction, when the reaction mixture was analyzed by high performance liquid chromatography (absolute calibration method), it was found that 0.89 g (Reaction yield; 71%) of 3-acetylthiophene was formed.

### Reference Example 5

### [R=Benzoyl Group, Y=Methoxyl Group]; Synthesis of 3-methoxy-1-phenylpropenone

In a flask having an inner volume of 500 ml and equipped with a stirrer, a thermomether and a reflux condenser were charged 16.2 g (0.30 mol) of sodium methoxide and 400 ml of diethyl ether. Then, while maintaining the liquid temperature to 10° C., a mixed solution comprising 39.7 g (0.33 mol) of acetophenone and 21.7 g (0.36 mol) of methyl formate was gradually added to the mixture, and the mixture was reacted under stirring at 25° C. for 3 hours. After completion of the reaction, the reaction mixture was filtered, and the filtrate was concentrated under reduced pressure to obtain 44.3 g of the concentrate. In a flask having an inner volume of 100 ml and equipped with a stirrer, a thermomether and a reflux condenser were charged 17.1 g of the concentrate, 11.4 g (82.5 mmol) of potassium carbonate, 70 ml of acetone and 12.36 g (98.0 mmol) of dimethylsulfate, and the mixture was reacted under stirring at 54 to 56° C. for 3 hours. After completion of the reaction, the reaction mixture was concentrated under reduced pressure, and the concentrate was distilled under reduced pressure (120 to 123° C., 667 Pa) to obtain 11.1 g (Isolation yield; 59%) of 3-methoxy-1-phenylpropenone as pale yellowish liquid.

Physical properties of the 3-methoxy-1-phenylpropenone are as follows.

¹H-NMR (CDCl₃, δ (ppm)); 3.82 (3H, s), 6.34 (1H, d, J=12.2 Hz), 7.40 to 7.60 (3H, m), 7.79 (1H, d, J=12.2 Hz), 7.80 to 7.92 (2H, m)

### Example 50

### [R=Benzoyl Group]; Synthesis of 3-benzoylthiophene

In a flask having an inner volume of 100 ml and equipped with a stirrer, a thermomether and a reflux condenser were charged 10.1 g (62.2 mmol) of 3-methoxy-1-phenylpropenone synthesized in the same manner as in Reference example 5, 4.30 g of 1,4-dithiane-2,5-diol (56.4 mmol as α-mercaptoacetaldehyde) and 60 ml of tetrahydrofuran, then, 4.3 g (28.8 mmol) of 1,8-diazabicyclo[5,4,0]-7-undecene and 10 ml of 1,2-dichloroethane were added to the mixture while maintaining the liquid temperature to 50° C., and the mixture was reacted under stirring at 67° C. for 3 hours. Then, 11 g (112 mmol) of 3 mol/l hydrochloric acid was added to the mixture, and the mixture was reacted under stirring at 60° C. for 2.5 hours. After completion of the reaction, the reaction mixture was concentrated under reduced pressure, the concentrate was extracted with 20 ml of ethyl acetate twice, and the extracts were concentrated. The obtained concentrate was purified by silica gel column chromatography (Eluent; n-hexane/ethyl acetate=5/1 to 1/1 (volume ratio)) to obtain 8.50 g (Isolation yield; 80%) of 3-benzoylthiophene as pale yellowish liquid.

Physical properties of the 3-benzoylthiophene are as follows.

¹H-NMR (CDCl₃, δ (ppm)); 7.38 (1H, dd, J=5.1, 2.9 Hz), 7.45 to 7.59 (3H, m), 7.61 (1H, dd, J=5.1, 1.2 Hz), 7.80 to 7.89 (2H, m), 7.93 (1H, dd, J=2.9, 1.2 Hz)

### Reference Example 6

### [R⁴=R⁵=methyl Group, M=Sodium Atom, n=1]; Synthesis of Sodium Salt of 1-hydroxy-4,4-dimethoxy-1-buten-3-one

In a flask having an inner volume of 1000 ml and equipped with a stirrer, a thermomether and a dropping funnel were charged 129.9 g (1.10 mol) of 1,1-dimethoxy-2-propanone and 370.4 g (5.00 mol) of ethyl formate, then, 192.9 g (1.00 mol) of a 28% sodium methoxide-methanol solution was gradually added to the mixture at room temperature, and the mixture was reacted at the same temperature for 24 hours. After completion of the reaction, the reaction mixture was concentrated under reduced pressure, 1240 ml of diethyl ether was added to the concentrate, and the mixture was stirred at room temperature for 30 minutes. After filtration, the filtrate was washed with diethyl ether and dried to obtain 91.9 g (Isolation yield; 49.2%) of a sodium salt of 1-hydroxy-4,4-dimethoxy-1-buten-3-one as ocherous powder with purity of 90% (Analytical value obtained by high performance liquid chromatography).

Physical properties of the sodium salt of 1-hydroxy-4,4-dimethoxy-1-buten-3-one are as follows.

¹H-NMR (CDCl₃, δ (ppm)); 3.19 (6H, s), 4.25 (1H, s), 4.79 (1H, d), 9.27 (1H, d)

### Reference Example 7

### [R⁴=R⁵=R⁶=Methyl Group]; Synthesis of 1,4,4-trimethoxy-1-buten-3-one

In a flask having an inner volume of 500 ml and equipped with a stirrer, a thermomether and a dropping funnel were charged 74.7 g (0.40 mol) of sodium salt of 1-hydroxy-4,4-dimethoxy-1-buten-3-one obtained in Reference example 5 with purity of 90%, 66.3 g (0.48 mol) of potassium carbonate and 160 ml of acetone, then, 60.5 g (0.48 mol) of dimethyl sulfate was gradually added to the mixture at room temperature, and the mixture was reacted at the same temperature for 24 hours. After completion of the reaction, the reaction mixture was filtered, and the filtrate was concentrated under reduced pressure to obtain 57.3 g (Isolation yield; 85.0%) of 1,4,4-trimethoxy-1-buten-3-one as dark brownish liquid with purity of 95% (Analytical value obtained by high performance liquid chromatography).

Physical properties of the 1,4,4-trimethoxy-1-buten-3-one are as follows.

¹H-NMR (CDCl₃, δ (ppm)); 3.42 (6H, s), 3.76 (3H, s), 4.59 (1H, s), 5.87 (1H, d), 7.82 (1H, d)

### Example 51

### [R=2,2-dimethoxyacetyl Group]; Synthesis of 3-(2,2-dimethoxyacetyl)-thiophene

In a flask having an inner volume of 300 ml and equipped with a stirrer, a thermomether and a dropping funnel were charged 53.4 g (0.30 mol) of 1,4,4-trimethoxy-1-buten-3-one obtained in Reference example 6 with purity of 90%, 22.8 g (0.15 mol) of 1,4-dithiane-2,5-diol and 75 ml of acetonitrile, then, 45.7 g (0.30 mol) of 1,8-diazabicyclo[5,4,0]-7-undecene was gradually added to the mixture at room temperature, and the mixture was reacted at the same temperature for 2 hours. After completion of the reaction, the filtrate was concentrated under reduced pressure, and ethyl acetate and water were added to the mixture and the organic layer was separated. The obtained organic layer was concentrated to obtain 39.4 g (Isolation yield; 60.0%) of 3-(2,2-dimethoxyacetyl)-thiophene as dark brownish liquid with purity of 85% (Analytical value obtained by high performance liquid chromatography).

Physical properties of the 3-(2,2-dimethoxyacetyl)-thiophene are as follows.

¹H-NMR (CDCl₃, δ (ppm)); 3.47 (6H, s), 5.02 (1H, s), 7.30 (1H, dd), 7.65 (1H, dd), 8.39 (1H, dd)

### UTILIZABILITY IN INDUSTRY

The 3-substituted thiophene obtained by the present invention is a useful compound as, for example, a synthetic intermediate or a starting material of a medicine, an agricultural chemicals, etc., or a synthetic starting material for chemicals for photography, and the like.

1. A process for preparing a 3-substituted thiophene represented by the formula (2): 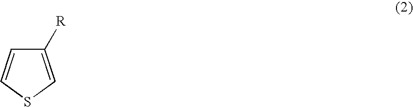
wherein R represents a cyano group, a formyl group, a carboxyl group, a hydrocarbyloxycarbonyl group which may have a substituent(s) or an acyl group which may have a substituent(s),
which comprises reacting a vinyl compound represented by the formula (1):
RCH═CHY (1)
wherein R has the same meaning as defined above, and Y represents a leaving group, and an α-mercaptoacetaldehyde or a multimer thereof.

2. The process for preparing a 3-substituted thiophene according to claim 1, wherein the leaving group Y of the vinyl compound represented by the formula (1) is a substituted amino group selected from a monoalkylamino group, monoarylamino group, dialkylamino group and diarylamino group; [NR¹R²R³]⁺X⁻ group wherein R¹ to R³ may be the same or different from each other, and each represent an alkyl group having 1 to 4 carbon atoms, a phenyl group or a benzyl group, and X represents a halogen atom; a halogen atom selected from a fluorine atom, chlorine atom, bromine atom and iodine atom; a substituted thio group selected from an alkylthio group and an arylthio group; an alkylsulfonyl group selected from a mesyl group; an arylsulfonyl group selected from a benzenesulfonyl group and tosyl group; an alkylsulfonyloxyl group selected from a methanesulfonyloxyl group and ethanesulfonyloxyl group; an arylsulfonyloxyl group selected from a benzenesulfonyloxyl group and p-toluenesulfonyloxyl group; an acyloxyl group having 1 to 6 carbon atoms selected from an acetoxyl group, propionyloxyl group and benzoyloxyl group; a hydrocarbyloxyl group which may have a substituent(s).

3. The process for preparing a 3-substituted thiophene according to claim 1, wherein the leaving group Y of the vinyl compound represented by the formula (1) is a dialkylamino group, a diarylamino group, a halogen atom and a hydrocarbyloxyl group which may have a substituent(s).

4. The process for preparing a 3-substituted thiophene according to claim 1, wherein the leaving group Y of the vinyl compound represented by the formula (1) is a dialkylamino group or a hydrocarbyloxyl group which may have a substituent(s).

5. The process for preparing a 3-substituted thiophene according to claim 1, wherein at least one additive selected from the group consisting of an organic base, an inorganic base, a metal alcoholate, an organic acid, a Lewis acid, a mineral acid, a cyclic polyether, a quaternary ammonium salt and a halogenated alkali is presented.

6. The process for preparing a 3-substituted thiophene according to claim 1, wherein at least one additive selected from the group consisting of an organic base, an inorganic base, a metal alcoholate, an organic acid, a Lewis acid, and a mineral acid is presented.

7. The process for preparing a 3-substituted thiophene according to claim 5, wherein an amount of the additive to be used is 0.01 to 10 mols based on 1 mol of the vinyl compound.

8. The process for preparing a 3-substituted thiophene according to claim 5, wherein an amount of the additive to be used is 0.02 to 5 mols based on 1 mol of the vinyl compound.

9. The process for preparing a 3-substituted thiophene according to claim 1, wherein an amount of the α-mercaptoacetaldehyde or a multimer thereof to be used is 0.2 to 20 mols in terms of the α-mercaptoacetaldehyde based on 1 mol of the vinyl compound.

10. The process for preparing a 3-substituted thiophene according to claim 1, wherein an amount of the α-mercaptoacetaldehyde or a multimer thereof to be used is 0.5 to 10 mols in terms of the α-mercaptoacetaldehyde based on 1 mol of the vinyl compound.

11. The process for preparing a 3-substituted thiophene according to claim 1, wherein R is selected from the group consisting of a cyano group, formyl group, methoxycarbonyl group, acetyl group, benzoyl group, valeryl group, octanoyl group, and 2,2-dimethoxy-acetyl group.

12. The process for preparing a 3-substituted thiophene according to claim 1, wherein the reaction is carried out by mixing the vinyl compound, the α-mercaptoacetaldehyde or a multimer thereof, and a solvent, and under stirring at −10 to 200° C.

13. The process for preparing a 3-substituted thiophene according to claim 1, wherein the reaction is carried out by mixing the vinyl compound, the α-mercaptoacetaldehyde or a multimer thereof, and a solvent, and under stirring at 0 to 150° C.
